(19) United States
(12) Patent Application Publication
(10) Pub. No.: US 2010/0256371 A1
(43) Pub. Date: Oct. 7, 2010

(54) PROCESSES FOR THE PREPARATION OF BOSENTAN AND ITS INTERMEDIATES THEREOF

(75) Inventors: Roop Singh Yadav, Navi Mumbai (IN); Mubeen Ahmed Khan, Navi Mumbai (IN); Sachin Mahadeo Lad, Thane (IN); Nikhil Rasiklal Trivedi, Navi Mumbai (IN); Sankar Arjunan, Navi Mumbai (IN); Gajendra Laxman Pitale, Ambernath (IN)

Correspondence Address: GLENMARK GENERICS INC., 750 CORPORATE DRIVE, MAHWAH, NJ, 07430, US

(73) Assignee: Glenmark, Mumbai (IN)

(21) Appl. No.: 12/724,430

(22) Filed: Mar. 16, 2010

Related U.S. Application Data
(60) Provisional application No. 61/228,661, filed on Jul. 27, 2009.

(30) Foreign Application Priority Data
Apr. 2, 2009 (IN) 902/MUM/2009
Jun. 10, 2009 (IN) 1398/MUM/2009

(51) Int. Cl. C07D 403/04

(52) U.S. Cl. 544/296

(57) ABSTRACT

The present invention relates to processes for the preparation of bosentan and compounds that can be used as structurally novel intermediates for the synthesis thereof, and a pharmaceutical composition of the same.

### PRIORITY

This application claims the benefit of priority under 35 U.S.C. §119 to U.S. provisional application No. 61/228,661 filed on Jul. 27, 2009 and Indian provisional application No. 902/MUM/2009 filed on Apr. 2, 2009, and Indian provisional application No. 1398/MUM/2009 filed on Jun. 10, 2009, the contents of which, are incorporated by reference herein.

### BACKGROUND OF THE INVENTION

1. Field of the Invention

The present invention relates to processes for the preparation of bosentan, novel compounds which can be useful intermediates for the synthesis thereof, and a pharmaceutical composition comprising the same.

2. Description of the Prior Art

Bosentan is an orally active dual endothelin ETA/ETB receptor antagonist and is used in the pulmonary arterial hypertension and systemic sclerosis. Bosentan is chemically described as 4-tert-butyl-N-[6-(2-hydroxyethoxy)-5-(2-methoxyphenoxy)-2-(2-pyrimidinyl)pyrimidin-4-yl]benzenesulphonamide monohydrate and has the structural formula, as shown

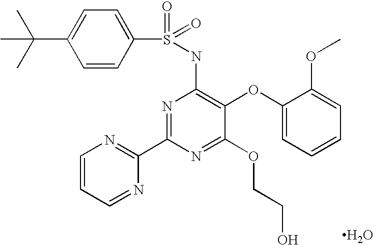

U.S. Pat. No. 5,292,740 (the '740 patent) describes pyrimidine sulfonamide derivatives including bosentan or a stereoisomer or salt thereof, a pharmaceutical composition and method of treatment.

The '740 patent discloses a process for the preparation of bosentan, which is illustrated by the scheme, which follows:

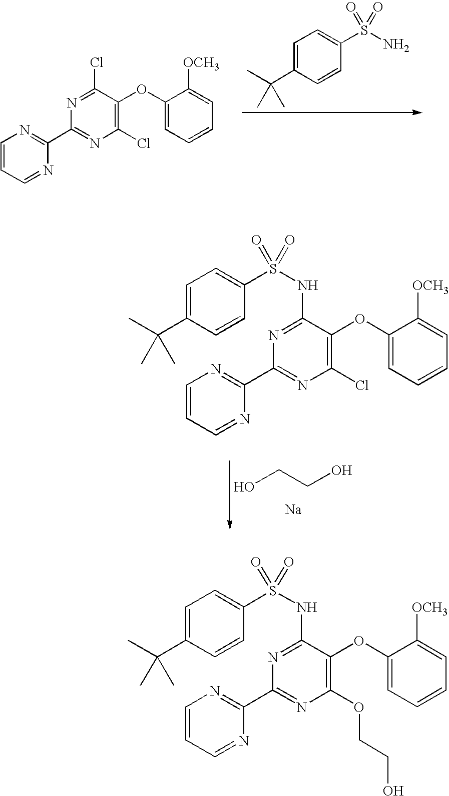

U.S. Pat. No. 6,136,971 describes a process for preparing ethylene glycol sulfonamide derivatives, including bosentan, using via formyl bosentan and pyrimidine monohalide intermediate, as illustrated by the scheme that follows:

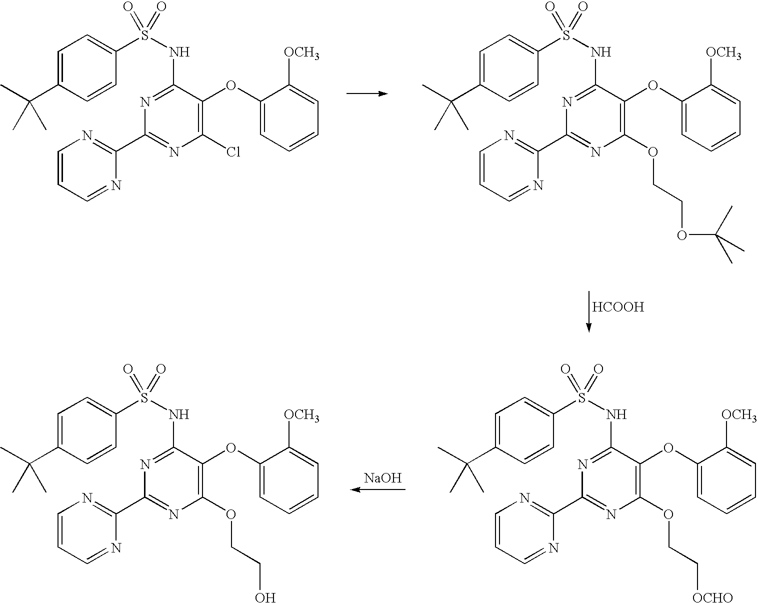

PCT Publication WO2009/004374 describes an improved process for the preparation of bosentan using sodium hydroxide for hydrolysis and via bosentan tartaric acid salt which is illustrated by the scheme, which follows:

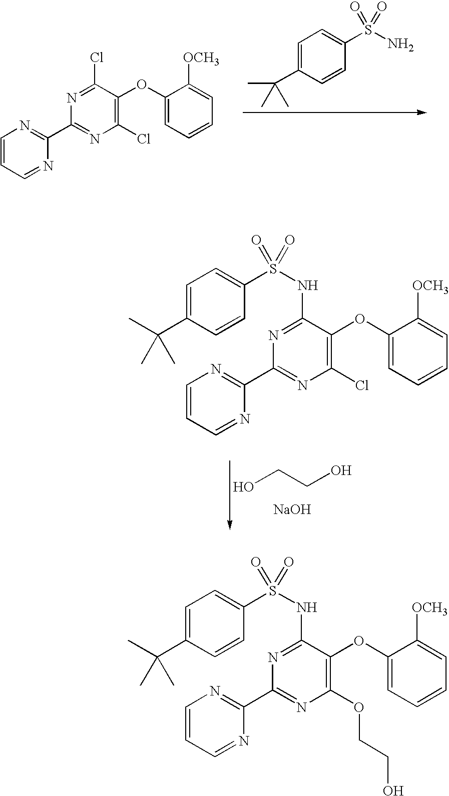

The present invention provides simple, ecofriendly, inexpensive, reproducible, robust processes for the preparation of bosentan and compounds useful as intermediates for the synthesis thereof, which are well suited on a commercial scale.

### SUMMARY OF THE INVENTION

The present invention relates to processes for the preparation of bosentan and compounds that can be used as novel intermediates for the synthesis thereof.

In one aspect, the present invention provides a process for preparing bosentan or a pharmaceutically acceptable salt thereof, comprising:

reacting 4-tertiarybutyl-N-[6-chloro-5-(O-methoxyphenoxy)[2,2′-bipyrimidin]-4-yl]benzenesulfonamide compound of formula I or a salt thereof

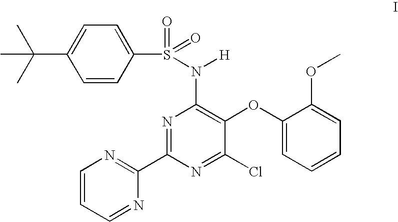

with ethylene glycol in the presence of alkoxide or hydride and an organic solvent to form bosentan.

The present invention provides a process for preparing bosentan comprising: reacting 4-tertiary butyl-N-[6-(2-alkoxyethoxy)-5-(2-methoxyphenoxy)-2-(2-pyrimidinyl)pyrimidin-4-yl]benzenesulfonamide compound of formula III or a salt thereof with a reducing agent in the presence of an organic solvent;

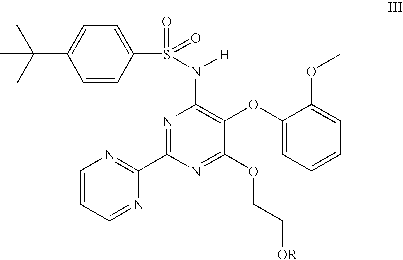

where R is methyl, ethyl, benzyl, hydrogen.

In yet another aspect, the present invention provides a process for preparing the compound of formula III or a salt thereof comprising: reacting 4-chloro-6-alkoxyethoxy-5-(O-methoxyphenoxy)-2,2′-bipyrimidine compound of formula IV

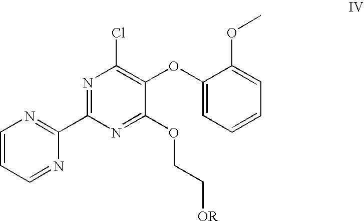

where R is same as defined previously for compound of formula III with 4-tertiarybutylphenylsulfonamide compound of formula V in the presence of a base and an organic solvent.

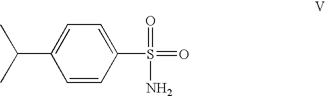

The present invention provides a process for preparing 4-chloro-6-alkoxyethoxy-5-(O-methoxyphenoxy)-2,2′-bipyrimidine compound of formula IV: comprising a) subjecting 4,6-dichloro-5-(O-methoxyphenoxy)-2,2′-bipyrimidine compound of formula VI

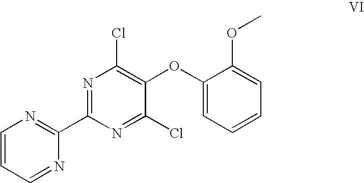

to basic hydrolysis to form 4-chloro-6-hydroxy-5-(O-methoxyphenoxy)-2,2′-bipyrimidine compound of formula VII

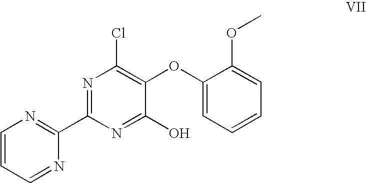

b) reacting the compound of formula VII with a compound of formula VIII in the presence of a base and an organic solvent under phase transfer conditions

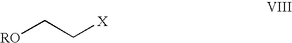

where R is benzyl, benzyloxy carbonyl, methyloxymethyl, hydrogen and X is halogen, OH.

In another aspect, the present invention provides a compound 4-chloro-6-alkoxyethoxy-5-(O-methoxyphenoxy)-2,2′-bipyrimidine of structural formula IV

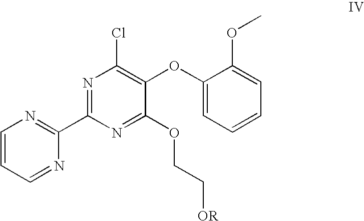

In yet another aspect, the present invention provides a compound 4-chloro-6-hydroxy-5-(O-methoxyphenoxy)-2,2′-bipyrimidine of structural formula VII

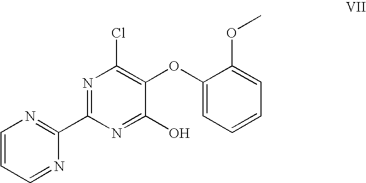

The present invention provides a process for the preparation of bosentan comprising reacting 4-tertiarybutyl-N-[6-(2-alkynoyloxyethoxy)-5-(2-methoxyphenoxy)-2-(2-pyrimidinyl)pyrimidin-4-yl]benzenesulfonamide compound of formula IX or a salt thereof

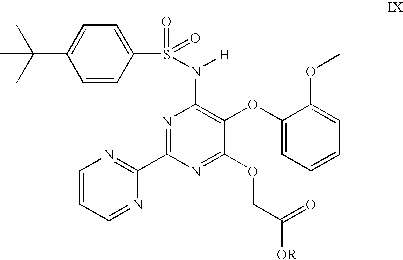

where R is H, methoxy, ethoxy

with a reducing agent in the presence of an organic solvent.

In still another aspect, the present invention provides a process for preparing a compound of formula IX comprising:
a) reacting 4-chloro-6-hydroxy-5-(O-methoxyphenoxy)-2,2′-bipyrimidine compound of formula VII

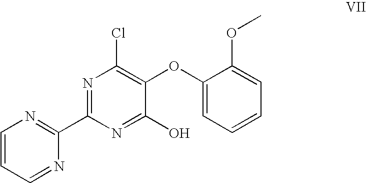

with a compound of formula X

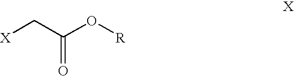

where R is same as defined for compound of formula IX and X is halogen atom. to form 4-chloro-6-alkanoyloxy ethoxy-5-(O-methoxyphenoxy)-2,2′-bipyrimidine compound of formula

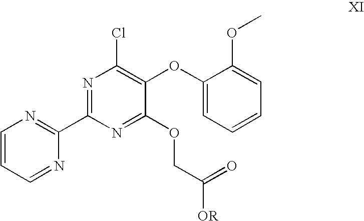

b) reacting the compound of formula XI with 4-tertiarybutylphenylsulfonamide compound of formula V or a salt thereof

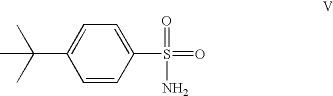

in the presence of a base and an organic solvent.

The present invention provides a compound 4-tertiarybutyl-N-[6-(2-alkynoyloxyethoxy)-5-(2-methoxyphenoxy)-2-(2-pyrimidinyl)pyrimidin-4-yl]benzene sulfonamide compound of formula IX or a salt thereof.

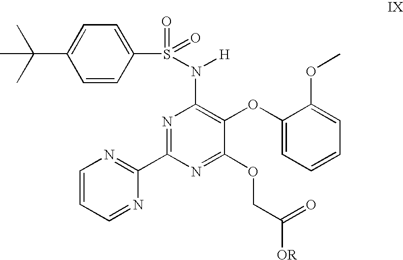

The present invention provides a compound 4-chloro-6-alkanoyloxy ethoxy-5-(O-methoxyphenoxy)-2,2′-bipyrimidine compound of structural formula XI

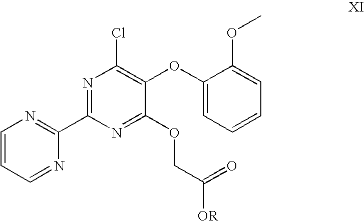

In yet still another aspect, the present invention provides a process for the purification of bosentan or pharmaceutically acceptable salt thereof comprising:
a) providing a solution of bosentan or salt thereof, prepared by the processes herein described, in a solvent or a mixture of solvents; and
b) optionally filtering the solution on celite; and
c) adding an anti-solvent to the filtrate to precipitate the solid;
d) recovering the solid to obtain bosentan in pure form.

The present invention provides bosentan or a pharmaceutically acceptable salt thereof, prepared by the processes herein described above, having less than about 0.5 area % of total impurities as measured by high performance liquid chromatography (HPLC).

The present invention provides bosentan or a pharmaceutically acceptable salt thereof, prepared by the processes herein described above, having less than about 0.15 area % of any individual impurity as measured by HPLC.

In another aspect, the present invention encompasses a pharmaceutical composition comprising bosentan or its pharmaceutically acceptable salts, prepared by processes herein described, and at least a pharmaceutically acceptable carrier.

### DETAILED DESCRIPTION OF THE INVENTION

The present invention provides a process for preparing bosentan or a pharmaceutically acceptable salt thereof comprising: reacting 4-tertiarybutyl-N-[6-chloro-5-(O-methoxyphenoxy)[2,2′-bipyrimidin]-4-yl]benzenesulfonamide compound of formula I or a salt thereof

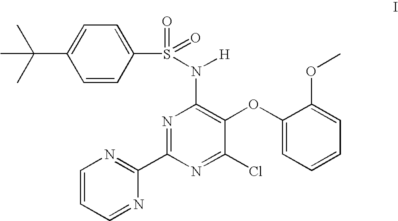

with ethylene glycol in the presence of an alkoxide or hydride and an organic solvent to form bosentan.

The alkoxide is selected from alkali metal alkoxide including sodium methoxide, sodium ethoxide, sodium isopropoxide, sodium tertiary butoxide, potassium methoxide, potassium ethoxide, potassium tertiary butoxide or alkaline earth metal alkoxide, as for example magnesium methoxide, magnesium ethoxide, magnesium tertiary butoxide and the like. Preferably the alkoxide is selected from sodium methoxide, sodium ethoxide, potassium methoxide. Most preferably the alkoxide is sodium methoxide.

The hydride is selected from alkali metal hydrides and alkaline earth metal hydrides include but are not limited to sodium hydride, potassium hydride, lithium hydride, magnesium hydride, calcium hydride and the like. Preferably, sodium hydride.

The molar equivalents of alkali or alkaline earth metal alkoxide or hydride employed is from about an equimolar amount to about 5 times the equimolar amount with respect to the compound of formula I.

The reaction is normally and preferably effected in the presence of an inert solvent. The solvents that can be used include but are not limited to haloalkanes such as dichloromethane, chloroform, carbon tetrachloride, dichloroethane and the like; ethers such as tetrahydrofuran, 1,4-dioxane, diisopropyl ether, methyl tertiary butyl ether and the like and mixtures thereof. Preferably tetrahydrofuran (THF).

The reaction can take place over a wide range of temperatures, from about 30° C. to about 100° C. Preferably from about 50° C. to about 55° C. More preferably from about 70° to about 80° C.

The time required for the reaction of preparing bosentan or a pharmaceutically acceptable salt thereof may also vary widely, depending on many factors, notably the reaction temperature and the nature of the reagents and solvents employed and volume of ethylene glycol. The reaction period may transpire from about 1 hour to about 20 hours. Preferably from about 5 hours to about 10 hours.

The dimer impurity depends on the volume of ethylene glycol in the reaction. Preferably the volume of ethylene glycol is from about 100 mole to about 110 mole of Formula I.

The reaction optionally can be carried out in neat conditions, i.e. in the absence of solvents.

The present invention provides a process for the preparation of 4-tertiary butyl-N-[6-hydroxy-5-(O-methoxyphenoxy)[2,2′-bipyrimidin]-4-yl]benzenesulfonamide compound of formula II or a salt thereof comprising: subjecting 4-tertiary butyl-N-[6-chloro-5-(O-methoxyphenoxy)[2,2′-bipyrimidin]-4-yl]benzenesulfonamide compound of formula I or a salt thereof

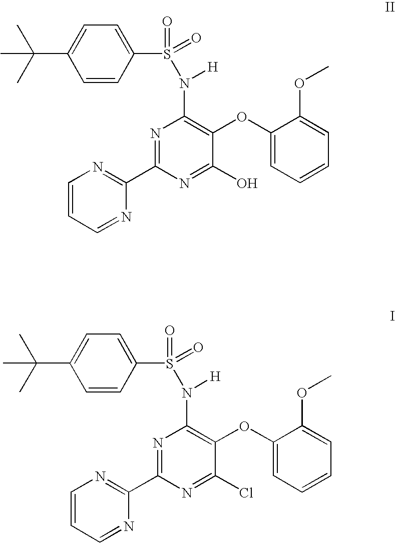

to aqueous basic hydrolysis.

The reaction is effected by using aqueous base and an inert solvent.

The bases that can be used include, but are not limited to, inorganic bases such as alkali metal alkoxides like sodium methoxide, sodium ethoxide, potassium t-butoxide and the like; alkali metal carbonates, such as sodium carbonate or potassium carbonate and the like; alkali metal hydroxides, such as sodium hydroxide, potassium hydroxide and the like. Of these, the alkali metal hydroxides are preferred. The amount of base employed is not critical, but good practice recommends an amount of base from about an equimolar amount to about 5 times the equimolar amount with respect to the compound of formula II.

The reaction is effected in the presence of a solvent. The solvents that can be used include, but are not limited to, halogenated solvent such as dichloromethane, ethylene dichloride, chloroform and the like; aprotic polar solvents such as N,N-dimethylformamide (DMF), dimethylsulfoxide (DMSO), dimethylacetamide (DMA), acetonitrile and the like; hydrocarbon solvents such as n-hexane, n-heptane, cyclohexane, toluene and the like and mixtures thereof. Preferably aqueous alcohols and polar aprotic solvents.

The reaction is carried out at a temperature from about 30° C. to about 100° C. or reflux temperatures of the solvents used. Preferably from about 30° C. to about 75° C.

The time required for the reaction may also vary widely, depending on many factors, notably the reaction temperature and the nature of the reagents and solvent employed. However, provided that the reaction is effected under the preferred conditions outlined above, a period from about 30 minutes to about 10 hours, preferably from about 30 minutes to about 5 hours, is sufficient. The molar ratio of the amounts of the compound of formula II and the base may be about 1:0.5 to about 1:5.

The present invention provides a process for preparing bosentan comprising reacting 4-tertiary butyl-N-[6-(2-alkoxyethoxy)-5-(2-methoxyphenoxy)-2-(2-pyrimidinyl)pyrimidin-4-yl]benzene sulfonamide compound of formula III or a salt thereof

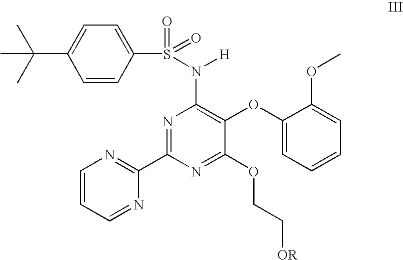

where R is benzyl, acetyl,
with a suitable reducing agent in the presence of an organic solvent.

The process of reduction is carried out by either using organic or inorganic acids, or using hydrogenation.

The inorganic acids that can be used include, but not limited to, sulfuric acid, hydrochloric acid, hydrobromic acid and the like; organic acids such as acetic acid, formic acid, trifluoroacetic acid and the like, preferably hydrochloric acid & sulfuric acid.

Alternatively, the process of reduction when carried out using hydrogenation, occurs with the use of hydrogenation catalysts in the presence hydrogen. The hydrogenation catalysts include but are not limited to palladium-carbon, Raney nickel and the like, preferably palladium-carbon.

The solvent that can be used is selected from alcohols such as methanol, ethanol, isopropyl alcohol and the like; esters such as ethyl acetate, isopropyl acetate and the like; halogenated solvents such as dichloromethane, ethylene dichloride, chloroform and the like; aprotic polar solvents such as N,N-dimethylformamide (DMF), dimethylsulfoxide (DMSO), dimethylacetamide (DMA), acetonitrile and the like; hydrocarbon solvents such as n-hexane, n-heptane, cyclohexane, toluene and the like and mixtures thereof. Preferably alcohols, more preferably, methanol.

The reaction can take place over a wide range of temperatures from about 0° C. to about 100° C. Preferably from about 0° C. to about 50° C.

The time required for the reaction can range from about 1 hour to about 20 hours. Preferably from about 1 hour to about 5 hours.

The present invention provides a process for preparing the compound of formula III or a salt thereof comprising: reacting4-chloro-6-alkoxyethoxy-5-(O-methoxyphenoxy)-2,2′-bipyrimidine compound of formula IV

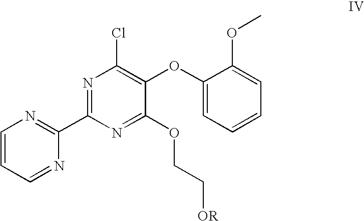

where R is benzyl, alkyl and hydrogen atom, as defined previously for compound of formula III

with 4-tertiarybutylphenylsulfonamide compound of formula V

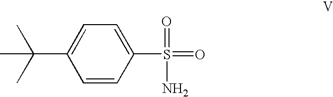

in the presence of a base and an inert organic solvent.

The reaction is carried out in the presence of a base and an inert solvent.

The bases that can be used include but are not limited to organic amines, such as triethylamine, tributylamine, N-methylmorpholine, pyridine, 4-dimethylamino-pyridine, lutidine, collidine and the like; alkali metal alkoxides, such as sodium methoxide, sodium ethoxide or potassium t-butoxide; alkali metal carbonates, such as sodium carbonate or potassium carbonate; and alkali metal hydroxides, such as sodium hydroxide or potassium hydroxide and mixtures thereof or metal hydrides. Preferably, sodium hydride.

The mole ratios of the compounds of formula IV and V would generally be from about an equimolar amount to about 5 times. Preferably, equimolar amounts.

The solvent that can be used is selected from alcohols such as methanol, ethanol, isopropyl alcohol and the like; esters such as ethyl acetate, isopropyl acetate and the like; halogenated solvents such as dichloromethane, ethylene dichloride, chloroform and the like; aprotic polar solvents such as N,N-dimethylformamide (DMF), dimethylsulfoxide (DMSO), dimethylacetamide (DMA), acetonitrile and the like; hydrocarbon solvents such as n-hexane, n-heptane, cyclohexane, toluene and the like and mixtures thereof. Preferably, aprotic polar solvents like DMF.

The reaction can take place over a wide range of temperatures from about 25° C. to about 150° C. Preferably from about 25° C. to about 75° C.

The time required for the reaction can range from about 1 hour to about 10 hours. Preferably from about 1 hour to about 5 hours.

The reaction of compound of formula IV with the compound of formula V can also be carried out in neat conditions in the absence of solvents.

The present invention provides a process for preparing 4-chloro-6-alkoxyethoxy-5-(O-methoxyphenoxy)-2,2′-bipyrimidine compound of formula IV comprising:
a) subjecting 4,6-dichloro-5-(O-methoxyphenoxy)-2,2′-bipyrimidine compound of formula VI

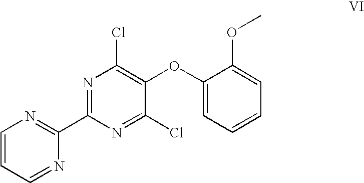

to basic hydrolysis to form 4-chloro-6-hydroxy-5-(O-methoxyphenoxy)-2,2′-bipyrimidine compound of formula VII

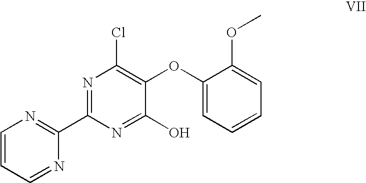

b) reacting the compound of formula VII with a compound of formula VIII in the presence of a base and an organic solvent under phase transfer conditions.

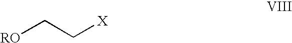

where R is benzyl, benzyloxy carbonyl, methyloxymethyl, H, and X is halogen, OH.

The bases that can be used include but are not limited to organic amines, such as triethylamine, tributylamine, N-methylmorpholine, pyridine, 4-dimethylamino pyridine, lutidine, collidine and the like; alkali metal alkoxides, such as sodium methoxide, sodium ethoxide or potassium t-butoxide; alkali metal carbonates, such as sodium carbonate or potassium carbonate; and alkali metal hydroxides, such as sodium hydroxide or potassium hydroxide and mixtures thereof or alkali metal hydride preferably alkali metal hydroxide.

The mole ratio of the compounds of formula VII and VIII would generally be from about an equimolar amount to about 5 times. Preferably, about 2 molars to about 3 molars.

The solvent that can be used is selected from alcohols such as methanol, ethanol, isopropyl alcohol and the like; esters such as ethyl acetate, isopropyl acetate and the like; halogenated solvents such as dichloromethane, ethylene dichloride, chloroform and the like; aprotic polar solvents such as N,N-dimethylformamide (DMF), dimethylsulfoxide (DMSO), dimethylacetamide (DMA), acetonitrile and the like; hydrocarbon solvents such as n-hexane, n-heptane, cyclohexane, toluene and the like and mixtures thereof. Preferably, alcohols.

The reaction can take place over a wide range of temperatures from about 25° C. to about 150° C. Preferably from about 25° C. to about 75° C.

The time required for the reaction can range from about 1 hour to about 10 hours. Preferably from about 1 hour to about 5 hours.

Optionally the compound of formula VI is reacted with the compound of formula VIII to give the compound of formula IV.

The present invention provides a compound: 4-chloro-6-alkoxyethoxy-5-(O-methoxyphenoxy)-2,2′-bipyrimidine of structural formula IV with R as hydrogen.

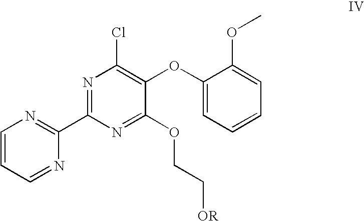

The present invention provides a process for the preparation of the compound IV with R as hydrogen comprising the reaction of 4,6-dichloro-5-(O-methoxyphenoxy)-2,2′-bipyrimidine compound of formula VI with ethylene glycol in the presence of base such as hydroxide ion bases and hydride ion bases. Preferably, sodium hydride.

The solvent that can be used is selected from alcohols such as methanol, ethanol, isopropyl alcohol and the like; esters such as ethyl acetate, isopropyl acetate and the like; halogenated solvents such as dichloromethane, ethylene dichloride, chloroform and the like; aprotic polar solvents such as N,N-dimethylformamide (DMF), dimethylsulfoxide (DMSO), dimethylacetamide (DMA), tetrahydrofuran (THF), acetonitrile and the like; hydrocarbon solvents such as n-hexane, n-heptane, cyclohexane, toluene and the like and mixtures thereof. Preferably, THF.

The present invention provides a compound: 4-chloro-6-hydroxy-5-(O-methoxyphenoxy)-2,2′-bipyrimidine of structural formula VII.

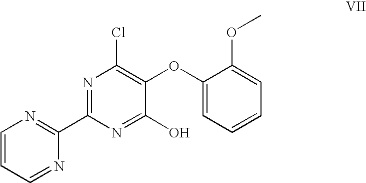

The present invention provides a process for the preparation of bosentan comprising: reacting 4-tertiarybutyl-N-[6-(2-alkynoyloxyethoxy)-5-(2-methoxyphenoxy)-2-(2-pyrimidinyl)pyrimidin-4-yl]benzene sulfonamide compound of formula IX or a salt thereof

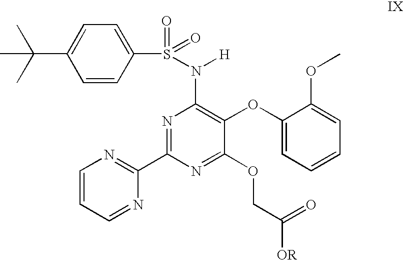

with a reducing agent in the presence of an organic solvent; where R is H, methoxy, ethoxy.

The process of reduction is carried out by hydrogenation.

The process of reduction is carried out using hydrogenation catalysts in the presence of hydrogen. The hydrogenation catalysts include but are not limited to palladium-carbon, Raney nickel and the like, preferably palladium-carbon.

The solvent that can be used is selected from the group alcohols such as methanol, ethanol, isopropyl alcohol and the like; esters such as ethyl acetate, isopropyl acetate and the like; halogenated solvents such as dichloromethane, ethylene dichloride, chloroform and the like; aprotic polar solvents such as N,N-dimethylformamide (DMF), dimethylsulfoxide (DMSO), dimethylacetamide (DMA), acetonitrile and the like; hydrocarbon solvents such as n-hexane, n-heptane, cyclohexane, toluene and the like and mixtures thereof. Preferably alcohols.

The reaction can take place over a wide range of temperatures from about 0° C. to about 100° C. Preferably, from about 0° C. to about 50° C.

The time required for the reaction can range from about 1 hour to about 20 hours. Preferably, from about 1 hour to about 5 hours.

The present invention provides a process for preparing a compound of formula IX comprising
a) reacting 4-chloro-6-hydroxy-5-(O-methoxyphenoxy)-2,2′-bipyrimidine compound of formula VII

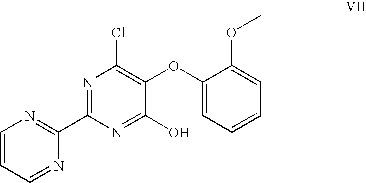

with a compound of formula X

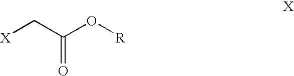

where R is same as defined for compound of formula IX and X is halogen atom, to form 4-chloro-6-alkanoyloxy ethoxy-5-(O-methoxyphenoxy)-2,2′-bipyrimidine compound of formula XI

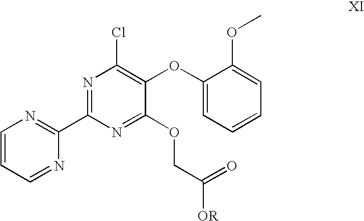

b) reacting the compound of formula XI with 4-tertiarybutylphenylsulfonamide compound of formula V or a salt thereof

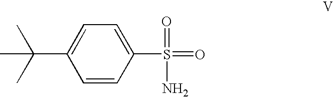

in the presence of a base and an organic solvent.

The reaction, previously described above, is carried out in the presence of a base and an inert solvent. The bases that can be used include but are not limited to organic amines, such as triethylamine, tributylamine, N-methylmorpholine, pyridine, 4-dimethylaminopyridine, lutidine, collidine and the like; alkali metal alkoxides, such as sodium methoxide, sodium ethoxide or potassium t-butoxide; alkali metal carbonates, such as sodium carbonate or potassium carbonate; and alkali metal hydroxides, such as sodium hydroxide or potassium hydroxide and mixtures thereof alkali metal hydride. Preferably sodium hydride.

The solvent that can be used is selected from alcohols such as methanol, ethanol, isopropyl alcohol and the like; esters such as ethyl acetate, isopropyl acetate and the like; solvents such as dichloromethane, ethylene dichloride, chloroform and the like; aprotic polar solvents such as N,N-dimethylformamide (DMF), dimethylsulfoxide (DMSO), dimethylacetamide (DMA), tetrahydrofuran (THF), acetonitrile and the like; hydrocarbon solvents such as n-hexane, n-heptane, cyclohexane, toluene and the like and mixtures thereof. Preferably, THF.

The reaction can take place over a wide range of temperatures from about 25° C. to about 150° C. Preferably, from about 25° C. to about 75° C.

The time required for the reaction can range from about 1 hour to about 24 hours. Preferably, from about 1 hour to about 5 hours.

The reactions, previously described, of a) a compound of formula VII with the compound of formula X; b) compound of formula XI with the compound of formula V can also be carried out in neat conditions.

The mole ratio of the compounds of formula VII and X employed would generally be from about an equimolar amount to about 5 times. Preferably, from about 2 molars to about 3 molars.

The present invention provides a compound 4-tertiarybutyl N-[6-(2-alkynoyloxyethoxy)-5-(2-methoxyphenoxy)-2-(2-pyrimidinyl)pyrimidin-4-yl]benzenesulfon amide compound of formula IX or a salt thereof.

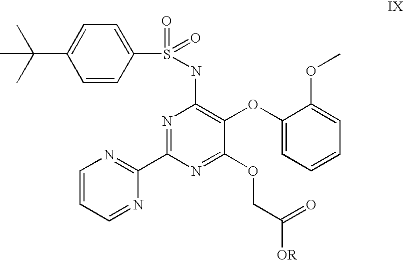

The present invention provides a compound 4-chloro-6-alkanoyloxy ethoxy-5-(O-methoxyphenoxy)-2,2′-bipyrimidine compound of structural formula XI

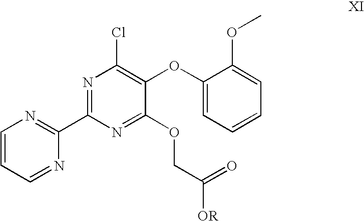

The isolation of the desired target compounds from the reaction mixtures in all of the above previously described processes can be carried out by methods known in the art Said methods take into consideration the physical properties of the desired compound, whereupon crystallization, extraction, washing, column chromatography, etc. may be combined. Preferably, extraction and crystallization.

The present invention provides that in the processes previously described, the intermediate compounds are optionally isolated and can be obtained by the so-called one pot reaction.

The target compound, bosentan, is optionally purified by recrystallisation using a solvent or mixture of solvents or alternatively, by converting into a pharmaceutically acceptable salt, which is subjected to purification then reverted back to bosentan.

In another aspect, the present invention provides a process for the purification of bosentan or pharmaceutically acceptable salt thereof comprising:
a) providing a solution of bosentan or pharmaceutically acceptable salt thereof in a solvent or a mixture of solvent;
b) optionally filtering the solution;
c) adding an anti-solvent to the filtrate to precipitate the bosentan or pharmaceutically acceptable salt thereof;
d) recovering the solid to obtain bosentan or pharmaceutically acceptable salt thereof in pure form.

The solvent that can be used is selected from the group consisting of ketones such as acetone, ethyl methyl ketone, methyl isobutyl ketone and the like; ethers such as tetrahydrofuran, 1,4-dioxane, diethyl ether, methyl tertiary butyl ether, diisopropyl ether, and the like and mixtures thereof. Preferably, acetone.

The temperatures for dissolution are from about 30 to about 75° C. or reflux temperatures of the solvents used. Preferably, from about 55° C. to about 60° C.

The solution obtained is optionally filtered on celite to remove the unwanted solids and particles.

The antisolvents that can be used include but are not limited to water, hydrocarbons such as n-pentane, n-hexane, n-heptane, cyclohexane and mixtures thereof. Preferably, water.

The temperature for precipitation of solid is from about 10° C. to about 35° C. Preferably from about 25° C. to about 30° C.

The obtained bosentan may be recovered by the conventional technique known in the art, preferably by filtration. The purification steps described above are optionally repeated.

Bosentan obtained is optionally dried at temperatures from about 35° C. to about 55° C. Preferably from about 50 to about 55° C. under vacuum of about 600 mmHg to about 710 mmHg.

In one embodiment, the present invention provides bosentan or a pharmaceutically acceptable salt thereof obtained by the processes described above having each one or none of the following impurities, at amounts determined by HPLC, namely:

Formula A 6-chloro-5-(2-methoxyphenoxy)-2,2′-bipyrimidin-4-ol.
Formula B 2-{[6-chloro-5-(2-methoxyphenoxy)-2,2′-bipyrimidin-4-yl]oxy}ethanol.
Formula C 6-hydroxy 2-sulfonamide impurity.
Formula D Bosentan-dimer impurity.
Formula E 6,6′-ethoxy-2-chloro impurity.
Formula F 6,6′-ethoxy-2-sulfonamide impurity
Formula G Desmethyl bosentan Impurity
Formula H Hydroxy desmethyl bosentan impurity
Formula I 6-Hydroxy bosentan Impurity
Formula J 6-Methoxy bosentan Impurity

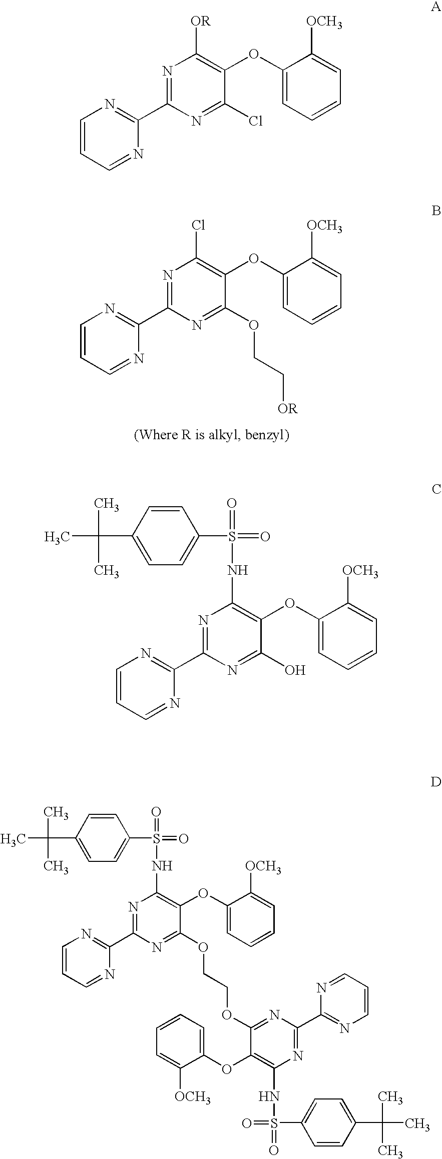

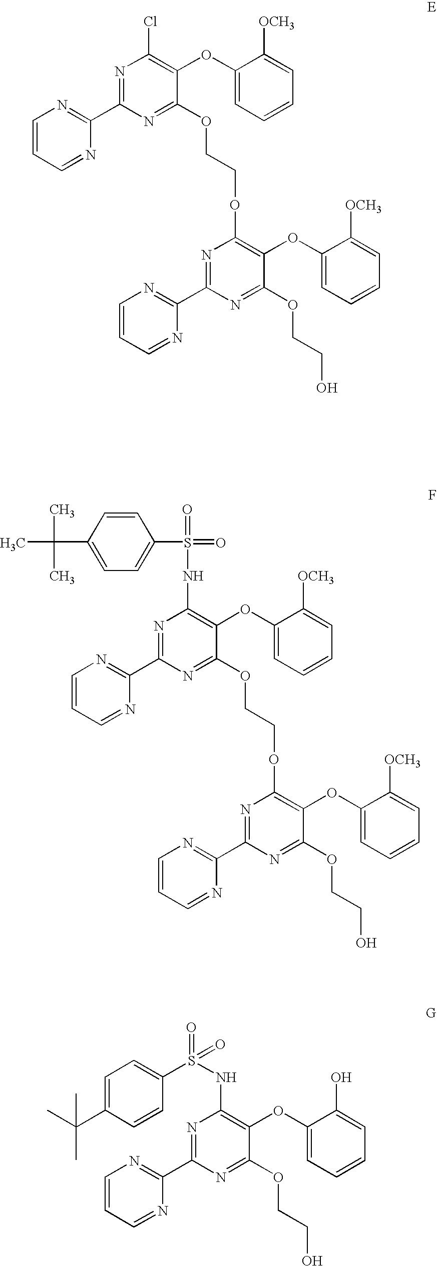

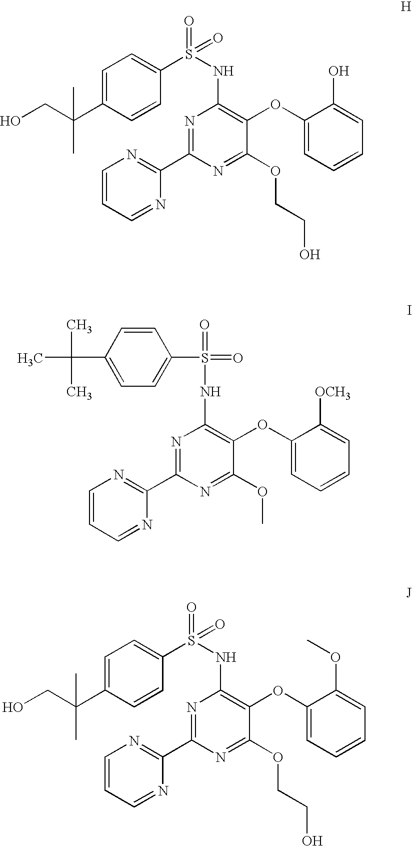

The present invention provides bosentan or a pharmaceutically acceptable salt thereof obtained by the above described processes, having any or each one the above described impurities in an amount less than about 0.15%.

The present invention provides bosentan or a pharmaceutically acceptable salt thereof obtained by the above described processes having a purity, as measured by HPLC, of at least about 98%, more preferably, at least about 99% and most preferably at least about 99.5%.

Preferably, the chemical purity of the bosentan or a pharmaceutically acceptable salt thereof is about 99% or more, more preferably about 99.5% or more, more preferably about 99.8% or more, more preferably about 99.9% or more, as measured by area under HPLC.

In one embodiment, the present invention encompasses bosentan or a pharmaceutically acceptable salt thereof having less than about 0.20% of any single impurity as measured by area under HPLC peaks. Preferably, the bosentan or a pharmaceutically acceptable salt thereof has less than about 0.15% of any single chemical impurity as measured by area under HPLC peaks.

The present invention provides a bosentan or a pharmaceutically acceptable salt thereof, obtained by the above process, as analyzed chemical purity using high performance liquid chromatography (“HPLC”) with the conditions described below:
Column: Zorbax SB-C₈, 250×4.6 mm, 5μ [Part No. 880975-906]
Column Temperature: 25° C.
Mobile phase: Mobile phase A=Buffer [Buffer: Dissolve 1 gm of Octane-1-sulfonic acid sodium salt in 1000 ml of water and mix. Add 1 ml of Triethylamine to it and mix. Adjust pH to 2.5 with diluted perchloric acid (10% in water)].
Mobile Phase B=Methanol

| Time(min.) | % Mobile Phase A | Mobile Phase B |
|---|---|---|
| 0.0 | 55 | 45 |
| 22 | 33 | 67 |
| 42 | 33 | 67 |
| 48 | 20 | 80 |
| 65 | 20 | 80 |
| 67 | 55 | 45 |
| 75 | 55 | 45 |

Diluent A: Water:Methanol:Methylene chloride (20:80:0.05, v/v/v)
Diluent B: Water: Methanol (20:80, v/v)
Flow Rate: 1.0 mL/minute
Detection: UV 220 nm
Injection Volume: 20 μL

In yet another embodiment, bosentan or its pharmaceutically acceptable salts obtained by the processes described above has residual organic solvents or organic volatile impurities less than the amount recommended for pharmaceutical products, as set forth for example in ICH guidelines and U.S. pharmacopoeia, which are less than about 600 ppm of dichloromethane, less than about 800 ppm of N,N-dimethylformamide (DMF), less than about 3000 ppm of methanol, 5000 ppm of acetone, ethyl acetate, isopropyl alcohol, and tetrahydrofuran (THF), less than about 890 ppm of toluene, Isopropyl acetate and acetic acid.

In one embodiment, bosentan or its pharmaceutically acceptable salts obtained by the process of the present invention can have a D₅₀ and D₉₀ particle size of less than about 400 microns, preferably less than about 200 microns, more preferably less than about 150 microns, still more preferably less than about 50 microns and most preferably less than about 15 microns. The particle size can be determined by such techniques as, for example, Malvern light scattering, a laser light scattering technique, etc., using, e.g., a Malvern® Mastersizer 2000. It is noted the notation Dₓ means that X % of the particles have a diameter less than a specified diameter D. Thus, a D₅₀ of about 250 μm means that 50% of the particles composition comprising of bosentan or its pharmaceutically acceptable salts have a diameter less than about 250 μm.

The particle sizes of the bosentan or its pharmaceutically acceptable salts obtained by, for example, milling, grinding, micronizing or other particle size reduction method known in the art to bring the solid state bosentan or its pharmaceutically acceptable salts to the desired the foregoing desired particle size range.

The present invention also provides pharmaceutical compositions comprising bosentan or its pharmaceutically acceptable salts obtained by the processes of present invention. Such pharmaceutical compositions may be administered to a mammalian patient in any dosage form, e.g., liquid, powder, elixir, injectable solution, etc. Dosage forms may be adapted for administration to the patient by oral, buccal, parenteral, ophthalmic, rectal and transdermal routes. Oral dosage forms include, but are not limited to, tablets, pills, capsules, troches, sachets, suspensions, powders, lozenges, elixirs and the like.

The simple, eco-friendly processes for the preparation of bosentan or its pharmaceutically acceptable salts thereof of the present invention are easily scalable.

The following examples are provided to enable one skilled in the art to practice the invention and are merely illustrative of the invention. The examples should not be read as limiting the scope of the invention as defined in the features and advantages.

### EXAMPLES

### Example 1

### PREPARATION OF 4,6-DIHYDROXY-5-(2-METHOXYPHENOXY)-2,2′-BIPYRIMIDINE

330.7 g (1.1811 mole) of 2-pyrimidine carboximidamide benzene sulphonate and 250 g (0.9842 mole) of 2,2-methoxy phenoxy melonate dimethyl ester were added to the clear solution of sodium methoxide at 40-45° C. (3 L of methanol and 132.87 g (2.4606 mole) of sodium methoxide). The reaction mixture was heated to reflux and maintained for about 5 hrs. Reaction was monitored by HPLC. Cooled the reaction mass up to about 40-45° C., then methanol was distilled off completely. The residue was taken into 2.5 L of purified water to get a clear solution and then added 212.5 ml of hydrochloric acid (Conc.) [pH of the mass ˜1.0] and stirred the reaction mass for about 4-5 hrs or until complete precipitation. The reaction mass was filtered and sucked dry. Washed with 1 L of purified water and finally washed with 125 ml of Acetone. The wet cake was charged in a clean flask and added 625 ml of acetone. The mass was stirred for about 3-4 hrs and the title compound, 4,6-Dihydroxy-5-(2-methoxyphenoxy)-2,2′-bipyrimidine, was filtered off and washed with 125 ml of acetone and dried in hot oven at about 50-55° C. until the LOD is below 10.0%.

Dry weight: 210 grms; Purity by HPLC: 98.8%.

### Example-2

### PREPARATION OF 4,6-DICHLORO-5-(2-METHOXYPHENOXY)-2,2′-BIPYRIMIDINE

120 g (0.3840 moles) of 6-Dihydroxy-5-(2-methoxyphenoxy)-2,2′-bipyridine was added to 235.38 g (1.5384 moles) of phosphorus oxychloride (POCl₃) under nitrogen and slowly added 75.96 g (0.9615 moles) of pyridine under nitrogen. The reaction mass was maintained for 30 minutes at the same temperature. The reaction mass was heated to about 95-100° C. for about 5 hrs. Reaction was monitored by HPLC. Cooled the reaction mass to about 10-15° C. and charged about 990 ml of methylene chloride to get a clear solution. Transferred the methylene chloride solution in another clean flask and added 990 ml of purified water and the mixture was cooled to about 5-10° C. The reaction mass was quenched in the mixture. Raised the temperature to about 25-30°. Stirred the mass for about 30 minutes and separated the product containing methylene chloride layer, extracted the aqueous layer with another 990 ml of Methylene chloride. The total methylene chloride layer was collected and washed with two portions of 990 ml each of purified water. Methylene chloride layer was washed with about 750 ml of 10% Sodium carbonate solution. Methylene chloride layer was washed with about 750 ml of 10% sodium chloride solution. The organic phase was dried using by sodium sulphate. The solvent was evaporated and the residue was treated with 990 ml of diisopropyl ether. The target compound, 4,6-Dichloro-5-(2-methoxyphenoxy)-2,2′-bipyrimidine was filtered off and washed with about 396 ml of Diisopropyl ether. Dried in hot air oven at about 50-55° C. for about 12-16 hrs until the LOD is about below 1.0%.

Dry weight 180 grms; Purity by HPLC: 99%.

### Example-3

### PREPARATION OF P-T-BUTYL-N-[6-CHLORO-5-(O-METHOXYPHENOXY)-4-PYRIMIDINYL]BENZENESULPHONAMIDE

66.0 g (0.3094 moles) of 4-t-butyl benzenesulphonamide, 53.38 g (0.3868 moles) of potassium carbonate, and 90 g (0.2578 moles) of 4,6-dichloro-5-(2-methoxyphenoxy)-2,2′-bipyrimidine were added to about 900 ml of toluene at about 25-30° C. Heat the reaction mass to reflux and maintain for about 3 hrs. Reaction was monitored by HPLC. Cooled the reaction mass to about 25-30° C. and charged about 900 ml of purified water into the reaction mass and stirred for about 60 min at about 25-30° C. Filtered the reaction mass and washed with about 450 ml of purified water. Unload the wet material and charged into the same flask. Charged about 900 ml of methylene chloride and about 900 ml of purified water and stirred the mass for about 60 minutes at about 25-30° C. Filtered the reaction mass and washed with about 450 ml of purified water. Unload the wet cake and charged in same flask. Charged about 900 ml of purified water and charged about 10 ml of 50% solution of HCl in to mass. Stirred the mass for about 60 minutes. Filtered the mass and washed with about 450 ml of purified water. Sucked dry completely. Charged the wet material into a flask and charged about 450 ml of acetone and 450 ml of purified water at about 25-30° C. Raised the temperature to about 60-65° C. and maintained the temperature for about 30 mins. Cooled the reaction mass to about 25-30° C. and maintained for about 30 minutes. Filtered and washed with a solvent mixture of about 90 ml each of acetone and purified water. Sucked dry completely. Unloaded the wet cake and dried under vacuum at about 50-55° C. for about 12-16 hrs until the water content was about below 3.5% % to give 112 gms of the title compound.

### Purity by HPLC: 98.5%.

### Example-4

### PREPARATION OF 4-TERT-BUTYL-N-[6-(2-HYDROXYETHOXY)-5-(2-METHOXYPHENOXY)-2-(2-PYRIMIDINYL) PYRIMIDIN-4-YL]BENZENESULFONAMIDE USING SODIUM METHOXIDE

630.0 g 1296.55 g (10.1711 moles 20.91 moles) of ethylene glycol and 51.33 g (0.9506 moles) of sodium methoxide were added to 1000 ml of tetrahydrofuran in a reaction vessel. The reaction mass was heated to about 50-55° C. to get a clear solution. 100 g (0.19011 moles) of p-t-butyl-N-[6-chloro-5-(o-methoxyphenoxy)-4-pyrimidinyl]benzenesulphonamide, prepared as in Example 3, was added in the reaction mass at about 50-55° C. The reaction mass was stirred for about 5-6 hrs for about 15-20 hrs at about 80-85° C. Reaction was monitored by TLC using 10% methanol and chloroform as mobile phase or alternatively, by HPLC. Cooled the reaction mass to about 40-45° C. The tetrahydrofuran was distilled off under vacuum at about 35-40° C. The resulting solution was cooled to 10-15° C. About 1000 ml each of dichloromethane and purified water were added into the resulting mass. The mass was stirred for about 30 min at about 10-15° C. The pH was adjusted to about 4.0-5.0 using 50% of hydrochloric acid. Stirred, settled, and separated the dichloromethane layer. Again extracted the aqueous layer using about 900 ml of dichloromethane. The total dichloromethane layers were combined and washed with three portions of 1000 ml each of purified water three times. The organic phase was dried by passing through sodium sulphate. Filtered the dried organic phase and then the solvent was evaporated to yield of 107 g of a foamy solid 4-tert-butyl-N-[6-(2-hydroxyethoxy)-5-(2-methoxyphenoxy)-2-(2-pyrimidinyl)pyrimidin-4-yl]benzene sulfonamide. Then the solid was added to a solvent mixture of about 360 ml each of methanol and isopropyl acetate. Heated the reaction mass to about 60-65° C. and cooled to about 25-30° C. and stirred for about 2 hrs at about the same temperature. The solid was filtered and washed with a solvent mixture of 90 ml each of methanol and isopropyl acetate to yield 80 g of the target compound, crude 4-tert-Butyl-N-[6-(2-hydroxyethoxy)-5-(2-methoxyphenoxy)-2-(2-pyrimidinyl)pyrimidin-4-yl]benzenesulfonamide.

### Yield: 87%; Purity by HPLC: 97.8%.

### Example-5

### PREPARATION OF 4-TERT-BUTYL-N-[6-(2-HYDROXYETHOXY)-5-(2-METHOXYPHENOXY)-2-(2-PYRIMIDINYL) PYRIMIDIN-4-YL]BENZENESULFONAMIDE USING SODIUM HYDRIDE

1166.9 g (18.821 moles) of Ethylene glycol, 34.2 g (1.425 moles) of Sodium hydride (60%) were added to 2100 ml of tetrahydrofuran. The reaction mass was heated to about 50-55° C. and stirred for about 2 hrs. 90.0 g (0.1710 moles) of p-t-butyl-N-[6-chloro-5-(o-methoxyphenoxy)-4-pyrimidinyl]benzenesulphonamide was added to the reaction mass. Heated the reaction mass to reflux and maintained for about 15-17 hrs at about 70-75° C. Reaction was monitored by HPLC. Cooled the reaction mass to about 25-30° C. and added about 2300 ml of methanol and the mixture was stirred for about 15 minutes. Charged 900 ml each of methylene chloride and of purified water into the reaction mass. The reaction mass was cooled to about 10-15° C. Added 196 ml of 50% of HCl solution and adjusted the pH to about 4.0-5.0 The solvent was distilled out under vacuum at about 40-45° C. The resulting solution was cooled to about 5-10° C. 2100 ml each of dichloromethane and of purified water were added into the resulting mass. The mass was stirred for about 30 minutes at about 5-10° C. pH was adjusted to about 3.5-4.5 using 50% of hydrochloric acid. Temperature was raised to about 25-30° C. and then separated the methylene chloride layer. Extracted the aqueous layer with about 2100 ml of methylene chloride. Combined total methylene chloride layers and washed with three portions of 2100 ml each of purified water. Methylene chloride layer was taken and washed with mixture of 1100 ml of methanol and 100 ml of purified water and was dried on sodium sulphate. Methylene chloride was distilled and degassed completely under vacuum. Charged 350 ml of methanol and 350 ml of isopropyl acetate and heated the reaction mass at 65° C. to get clear solution an stirred at same temp. for 15-30 mins. Cooled the reaction mass to 25-30° C. and maintained for 2 hrs at 25-30° C. Filtered the product and washed with mixture of 175 ml of methanol and 175 ml of Isopropyl acetate. Suck dried completely. Bosentan wet wt: 145 grms; Dry wt: 78 grms; Purity by HPLC: 98.8%.

The resulting mother liquor was cooled to about 5-10° C. 10 V each of dichloromethane and of purified water were added into the resulting mass. The mass was stirred for about 30 minutes at about 5-10° C. The solution's pH was adjusted to about 3.5-4.5 using 50% of hydrochloric acid. Stirred, settled, and separated the dichloromethane layer. Again, extracted the aqueous layer with the use of another 10 V of dichloromethane. The total dichloromethane layers were combined and washed with three portions about 5 V of each purified water. The organic phase was dried using sodium sulphate. Filtered and evaporated the solvent to yield of 10.5 g of foamy solid 4-tert-butyl-N-[6-(2-hydroxyethoxy)-5-(2-methoxyphenoxy)-2-(2-pyrimidinyl)pyrimidin-4-yl]benzene sulfonamide. 10 V of diisopropyl ether was added and then stirred for about 4-6 hrs. The solid was filtered and washed with diisopropyl ether to yield 10 g of crude 4-tert-Butyl-N-[6-(2-hydroxyethoxy)-5-(2-methoxyphenoxy)-2-(2-pyrimidinyl)pyrimidin-4-yl]benzene sulfonamide; Purity by HPLC: 97.8%.

### Example-6

### Purification of Bosentan Using Acetone and Water

10 g of bosentan was added to 30 ml of acetone and refluxed to get a clear solution. 20 ml of water was added dropwise at reflux temperature. The suspension was slowly cooled to about 25-30° C. Filtered the mass and dried at about 25-30° C. under vacuum. Dry weight 8.9 grms; Purity by HPLC: 99.6%.

### Example-7

### Purification of Bosentan Using Ethanol and Water

10 g of bosentan was added to 30 ml of ethanol and refluxed to get a clear solution. 30 ml of Water was added dropwise at reflux temperature. The suspension was slowly cooled to about 25-30° C. Filtered the mass and dried at about 25-30° C. under vacuum. Dry weight: 9 grms, Purity by HPLC: 99.6%.

### Example-8

### Purification of Bosentan Using Methanol

10 g of bosentan was added to 100 ml of methanol and refluxed to obtain a clear solution. The clear mass was stirred and slowly cooled to about 25-30° C. and filtered. The solid product was dried at about 25-30° C. under vacuum for about 10-12 hrs. Dry weight: 9 grms; Purity by HPLC: 98.9%.

### Example-9

### Purification of Bosentan Using Acetonitrile and Water

10 g of bosentan was added to 40 ml of acetonitrile and refluxed to obtain a clear solution. 30 ml of Water was added dropwise at reflux temperature. The suspension was slowly cooled to 25-30° C. Filtered the mass and dried at about 25-30° C. under vacuum. Dry weight: 8.9 grms; Purity by HPLC: 99.5%.

### Example-10

### Purification of Bosentan Using Acetonitrile

10 g of bosentan was added to 30 ml of acetonitrile and refluxed to obtain a clear solution. The clear mass was stirred and slowly cooled to about 25-30° C. and filtered. The solid product was dried at about 25-30° C. under vacuum for about 10-12 hrs. Dry weight: 8.7 grms; Purity by HPLC: 98.9%

### Example-11

### Purification of Bosentan Using THF and Water

10 g of Bosentan was added to 20 ml of tetrahydrofuran and refluxed to get a clear solution. 30 ml of water was added dropwise at reflux temperature. The suspension was slowly cooled to about 25-30° C. Filtered the mass and dried at about 25-30° C. under vacuum.

Dry weight: 9 grms; Purity by HPLC: 98.9%.

1. A process for the preparation of bosentan or a pharmaceutically acceptable salt thereof comprising: 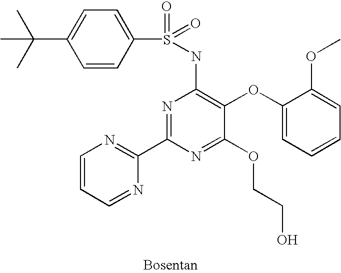
reacting 4-tertiary butyl-N-[6-chloro-5-(O-methoxyphenoxy)[2,2′-bipyrimidin]-4-yl]benzene sulfonamide compound of formula I or a salt thereof 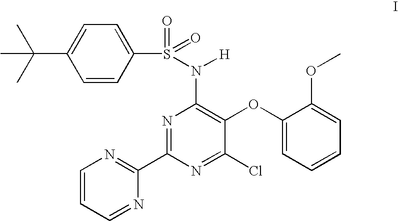
with ethylene glycol in the presence of an alkoxide or a hydride and an organic solvent to form bosentan or a pharmaceutically acceptable salt thereof.

2. The process of claim 1, wherein the alkoxide is selected from alkali metal alkoxide comprising sodium methoxide, sodium ethoxide, sodium isopropoxide, sodium tertiary butoxide, potassium methoxide, potassium ethoxide, potassium tertiary butoxide or alkaline earth metal alkoxide comprising magnesium methoxide, magnesium ethoxide, magnesium tertiary butoxide.

3. The process of claim 1, wherein the hydride is selected from alkali metal hydrides comprising sodium hydride, potassium hydride or alkaline earth metal hydrides comprising magnesium hydride, calcium hydride.

4. The process of claim 1, wherein the organic solvent is selected from haloalkanes solvents comprising dichloromethane, chloroform, carbon tetrachloride, dichloroethane; ethers comprising tetrahydrofuran, 1,4-dioxane, diisopropyl ether, methyl tertiary butyl ether and mixtures thereof.

5. Bosentan or a pharmaceutically acceptable salt thereof having a purity of greater than about 99.5 area % as measured by high performance liquid chromatography.

6. Bosentan or a pharmaceutically acceptable salt thereof, prepared by the process of claim 1, having less than about 0.5 area % of total impurities as measured by high performance liquid chromatography.

7. Bosentan or a pharmaceutically acceptable salt thereof, prepared by the process of claim 1, having less than about 0.15 area % of any individual impurity as measured by high performance liquid chromatography.
